(19) United States
(12) Patent Application Publication
(10) Pub. No.: US 2023/0250069 A1
(43) Pub. Date: Aug. 10, 2023

(54) AN INDUSTRIAL SCALE PROCESS FOR THE PREPARATION OF PROTHIOCONAZOLE

(71) Applicant: Hikal Limited, Maharashtra, Pune (IN)

(72) Inventors: Sudhir NAMBIAR, Maharashtra, Pune (IN); Santosh GHOSH, Maharashtra, Pune (IN); Nagaprasada Rao L., Maharashtra, Pune (IN); Pramod SAWANT, Maharashtra, Pune (IN); Vinayak LAMBATE, Maharashtra, Pune (IN); Dnyaneshwar NIPUNGE, Maharashtra, Pune (IN); Somwanshi ATUL, Maharashtra, Pune (IN)

(73) Assignee: Hikal Limited, Maharashtra, Pune (IN)

(21) Appl. No.: 17/766,662

(22) PCT Filed: Oct. 5, 2020
(86) PCT No.: PCT/IB2020/059331

§ 371 Date: Apr. 5, 2022

(30) Foreign Application Priority Data
Oct. 17, 2019 (IN) 201921042108

(51) Int. Cl. C07D 249/12

(52) U.S. Cl. CPC C07D 249/12

(57) ABSTRACT

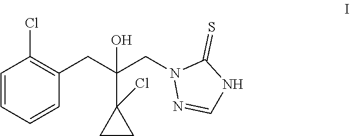 [SMILES: OC(Cc1ccccc1Cl)(Cn1nc[nH]c1=S)C1(Cl)CC1]

The present invention relates to an industrial scale process for the preparation of Prothioconazole (I), which is simple, economical, efficient, user and environment friendly, moreover commercially viable with higher yield and greater chemical purity.

### RELATED APPLICATION

This application claims the benefit to Indian Provisional Application No. 201921042108, filed on Oct. 17, 2019, the contents of which are incorporated by reference herein.

### FIELD OF THE INVENTION

The present invention relates to an industrial scale and efficient process for the preparation of Prothioconazole of formula (I). The process produces Prothioconazole in high yield with greater chemical purity in an environment friendly and commercially viable manner.

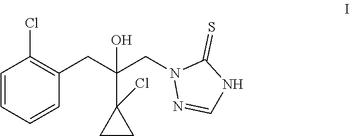 [SMILES: OC(Cc1ccccc1Cl)(Cn1nc[nH]c1=S)C1(Cl)CC1]

### BACKGROUND OF THE INVENTION

Prothioconazole, 2- [2-(1-Chlorocyclopropyl) -3-(2-chlorophenyl) -2-hydroxypropyl]-2, 4-dihydro-3H-1, 2, 4-triazole-3-thione (I) is a broad spectrum anti-fungal agent of triazolinthione family and is used as a fungicide to treat infected crops especially in cereals. Prothioconazole was first disclosed in U.S. Pat. No. 5,789,430 and corresponding patent publications, as a triazolyl derivative.

The references predominantly disclose the synthesis of Prothioconazole in two ways, using (i) elemental sulfur (powder) as a sulfur source for Prothioconazole synthesis which generally involve hydroxy triazole compound as a key intermediate reacting with sulfur to obtain Prothioconazole; and (ii) thiocyanate as a sulfur source. Both routes involve variety of reagents, reactants and have some limitations. U.S. Pat. No. 5,789,430 (henceforth ′430) discloses the preparation of Prothioconazole by reaction of a 2-(1-chloro-cyclopropyl)-1-(2-chlorophenyl)-3-(1, 2,4-triazol-1-yl)-propan-2-ol with sulfur powder in absolute N-methyl-pyrrolidone at 200° C. for 44 hours gives only 20% yield and with n-BuLi in tetrahydrofuran which gives good yield. However, use of both these processes would result in the production of regioisomeric impurities.

U.S. Pat. No. 4,913,727 discloses preparation of (1, 2,4-triazol-1-yl -methyl)-(1-chloro cycoprop-1-yl)-ketone by reaction of 1-chloro-2-(1-chlorocyclopropyl)-3-(2-chlorophenyl) propan-2-ol with 1,2,4-triazole under basic condition. The disadvantage of this process is that under basic condition, 1,2,4-triazole undergoes isomerization, resulting in the formation of corresponding regioisomer impurity.

U.S. Pat. No. 6,262,276 disclose the second strategy of producing Prothioconazole using thiocyanate through thiosemicarbazide where thiosemicarbazide in isobutyl formate is admixed with formic acid. However, the purity of thiosemicarbazide (65.9%) is not favorable for subsequent step, it may be because of the formation of regioisomeric impurity. Another reference U.S. Pat. No. 6,271,389 discloses a method of preparing Prothioconazole using potassium thiocyanate by reacting with 2-(1-chloro-cyclopropyl)-3-(2-chloro-phenyl)-2-hydroxy-propyl-1-hydrazine to produce triazolidinethione derivative which is further treated with formic acid and isobutyl formate to produce Prothioconazole. However, the overall yield is less as it involves an additional deprotection step.

Therefore, there is a need for an improved process for preparation of Prothioconazole which reduces the impurities, decreases the number of steps, using safe reagents to enable more robust process, which is industrial friendly and economically viable. The inventors of instant application motivated to develop an efficient process for Prothioconazole using thiocyanate starting from 2-acetylbutyrolactone in fewer number of steps (three steps) with simple isolation process, which is resulting in high chemical yield, greater purity and low effluent.

### OBJECT OF THE INVENTION

The main object of the present invention is to provide an industrial scale and efficient process for the preparation of Prothioconazole of formula (I) which is simple, economical, user-friendly and commercially viable.

Another objective of the process of present invention is to obtain high yield and greater chemical purity of Prothioconazole of formula (I).

Yet another objective of the present invention is to provide an industrial scale process for the preparation of Prothioconazole of formula (I) in fewer numbers of steps, thus reduces overall cycle time.

Yet another objective of the present invention is to provide a cost-effective process by reducing usage of equipment(s) in commercial scale.

### SUMMARY OF THE INVENTION

In one aspect the present invention provides a process for the preparation of

Prothioconazole of formula (I), which comprises the steps:

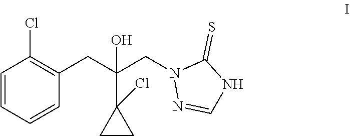 [SMILES: OC(Cc1ccccc1Cl)(Cn1nc[nH]c1=S)C1(Cl)CC1]

1) obtaining compound of formula (III) from a compound of formula (II) by sequential reaction transformations;

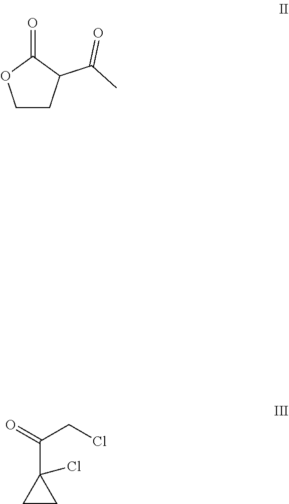

2) obtaining compound of formula (VI) by

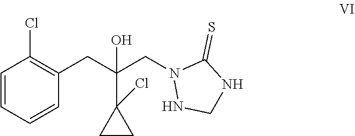 [SMILES: OC(Cc1ccccc1Cl)(CN1NCNC1=S)C1(Cl)CC1]

a) reacting compound of formula (III) with Grignard reagent of formula (IVa), where X is Cl, Br, or I, in solvent to obtain a mixture of oxirane of formula (Va) and alcohol of formula (Vb) at suitable temperature;

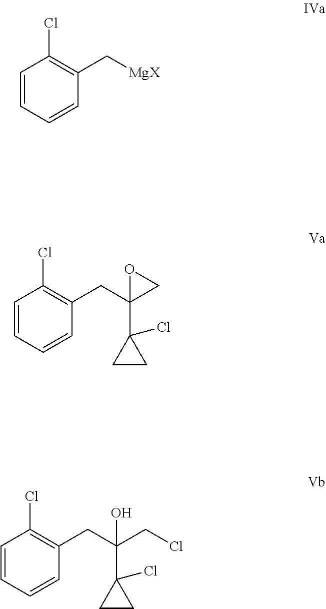

b) reacting mixture of formula (Va) and formula (Vb) with hydrazine hydrate in solvent; followed by reacting with formaldehyde, thiocyanate of formula YSCN, where Y is sodium, potassium or ammonium, in presence of acid and water; and

3) obtaining compound of formula (I) by oxidation of compound of formula (VI) in suitable solvent, optionally the compound is purified.

In another aspect, in the preparation of Prothioconazole of formula (I), the step 1 involves sequentially chlorination, hydrolysis, cyclization and chlorination.

### DETAILED DESCRIPTION OF THE INVENTION

The present invention now will be described more fully hereinafter. The invention may be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly indicates otherwise.

In accordance with the objectives, the present invention provides an industrial scale process for the preparation of Prothioconazole of formula (I).

In one embodiment, the instant invention provides the preparation of Prothioconazole which involve three steps starting from 2-acetylbutyrolactone, thus the process is economically viable.

In another embodiment, the process for preparation of Prothioconazole generates less effluent and thus the process is environment friendly, safer and thereby commercially viable.

The process for the preparation of Prothioconazole is illustrated in the following general synthetic scheme:

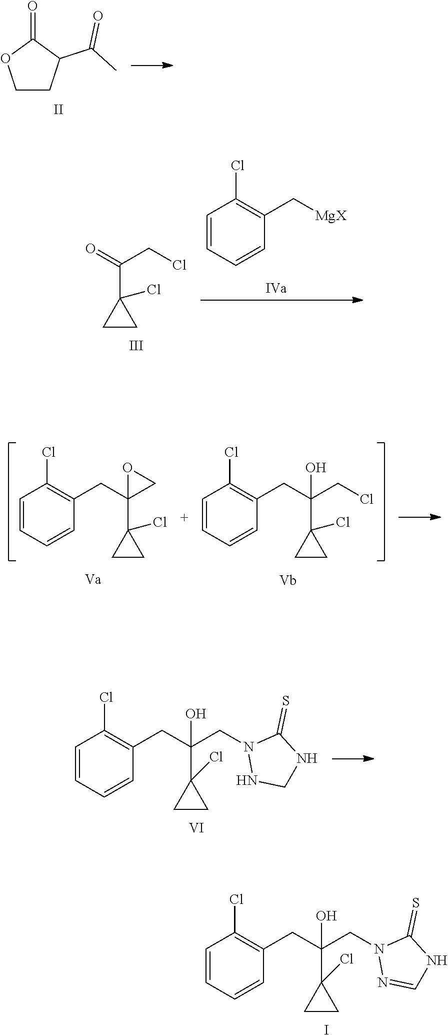

In another embodiment, the present invention provides a process for the preparation of Prothioconazole of formula (I) with purity greater than 98%, preferably 99%.

In another embodiment, the present invention provides an improved process for the preparation of compound of formula (III) from compound of formula (II) which is having purity greater than 92%.

In another embodiment of present invention, wherein the chlorinating agent in step (1) is selected from the group consisting of sulfuryl chloride (SO₂Cl₂); chlorine gas (Cl₂ gas); chlorine gas in presence of sodium acetate (AcONa) in acetic acid (AcOH); thionyl chloride (SOCl₂); N-chloro succinimide (NCS); cyanuric chloride [(NCCl)₃]; 1,3-dichloro 5,5-dimethylhydantoin; oxidative chlorinating agents; oxalyl chloride [(COCl)₂], phosphoryl chloride (POCl₃), phosphorus pentachloride (PCl₅), phosphorus trichloride (PCl₃) and the like.

The term solvent used herein, refers to the single solvent or mixture of solvents.

In another embodiment of present invention, the chlorination in step (1) is carried out with or without solvent.

In another embodiment of present invention, wherein solvent in step (1) is selected from dichloromethane (DCM), ethylene dichloride (EDC), chloroform (CHCl₃), carbon tetrachloride (CCl₄), toluene, cyclohexane, monohalobenzenes such as monochlorobenzene, dihalobenzenes such as dichlorobenzene, dialkyl (C₁-C₁₂) ethers, water and the like.

In another embodiment of present invention, wherein the chlorination of step (1) is carried out with or without catalytic amount of alcohol which is selected from lower alkyl alcohols preferably C₁-C₄ alcohols; in this embodiment alcohol is used in 0.1 to 3.0 equivalents.

In another embodiment of present invention, wherein the C₁-C₄ alcohols used in chlorination reaction is selected from methanol, ethanol, isopropanol, n-butanol and the like.

In another embodiment of present invention, wherein the chlorination of step (1) reduces the formation of geminal dichloro impurity in presence of alcoholic solvent.

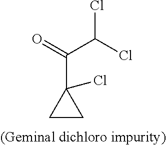

In another embodiment of the present invention, wherein the hydrolysis in step (1) is carried out with acid for example hydrochloric acid (HCl), hydrobromic acid (HBr), in presence or absence of another acid selected from the group consisting of sulfuric acid, trifluoroacetic acid (TFA), formic acid (HCOOH), and acetic acid (AcOH).

In another embodiment of the present invention, wherein the acid hydrolysis in step (1) is carried out at a temperature between 40° C. to 110° C., preferably 70° C. to 100° C.

In another embodiment of the present invention, wherein the cyclization in step (1) is carried out by using base in presence or absence of phase transfer catalyst.

In another embodiment of the present invention, wherein the cyclization in step (1) is carried out by using base selected from the group consisting of sodium hydroxide (NaOH), potassium hydroxide (KOH), potassium carbonate (K₂CO₃), sodium carbonate (Na₂CO₃), ammonia (NH₃), ammonium hydroxide (NH₄OH), magnesium tertiary butoxide [(t-BuO)₂Mg], potassium tertiary butoxide (t-BuOK), and sodium tertiary butoxide (t-BuONa).

In another embodiment of the present invention, wherein the cyclization in step (1) is carried out by using base preferably using 10% to 50% with or without phase transfer catalyst in concentrations of 0.005 equivalents to 0.015 equivalents.

In another embodiment of the present invention, wherein the phase transfer catalyst in cyclization of step (1) is selected from the group consisting of tetra alkyl ammonium halide such as tetrabutylammonium bromide (TBAB), tetrabutylammonium iodide (TBAI), tetrabutylammonium chloride (TBACl), tetrabutylammonium fluoride (TBAF), benzyltriethylammonium chloride; methyl tri alkyl ammonium halides such as methyltricaprylammonium chloride, methyltributylammonium chloride, methyl trioctylammonium chloride, Aliquat 336; and potassium iodide (KI).

In another embodiment of the present invention, wherein the step (1) is carried out at temperature between 20° C. to 50° C.

In another embodiment of the present invention, wherein the solvent in step (2a) is a mixture of tetrahydrofuran (THF) and toluene, inert organic solvents such as aliphatic, alicyclic and aromatic hydrocarbons solvent selected from cyclohexane, methylcyclohexane, xylene, benzene, 2-methyltetrahydrofuran, methyl tert-butyl ether, isopropyl ether, dimethoxyethane, dimethoxymethane, 1,3-dioxane, 1,4-dioxane, ethylene glycol diethyl ether, diethylene glycol dimethyl ether, diethylene glycol diethyl ether, diethylene glycol dibutyl ether, polyethylene glycol dimethyl ether, cyclic and acyclic ethers of one, several or a mixture in any proportions.

In another embodiment of the present invention, wherein the suitable solvent in step (2a) is a mixture of tetrahydrofuran (THF) and toluene in ratio of between 35:65 and 5:95.

In another embodiment of the present invention, wherein the Grignard reagent of formula (IVa) in step (2a) is prepared by reacting 2-chlorobenzylchloride or 2-chlorobenzylbromide or 2-chlorobenzyliodide with magnesium (Mg) in presence or absence of initiator.

In another embodiment of the present invention, wherein the initiator used in step 2(a) is selected from Iodine, methyl iodide and 1,2-dibromoethane.

In another embodiment of the present invention, wherein the Grignard reagent of formula (IVa) in step (2a) is used in between 1.2 to 1.4 equivalents which favors the formation of oxirane of formula (Va) under heating up to 70-80%.

In another embodiment of the present invention, wherein the suitable temperature in step (2a) is −5° C. to 80° C.

In another embodiment of the present invention, wherein the solvent in step (2b) is selected from the group consisting of alcohol such as lower alkyl alcohols preferably C₁-C₄ alcohols, cyclohexanol, toluene, acetonitrile (ACN), N, N-dimethyl formamide (DMF) and sulfolane.

In another embodiment of the present invention, wherein the equivalents of hydrazine hydrate in step (2b) is between 1.2 to 10 equivalents preferably 5 equivalents.

In another embodiment of the present invention, wherein the acid used in step (2b) is selected from the group consisting of sodium hydrogen sulfate (NaHSO₄), p-toluenesulfonic acid (p-TSA), acetic acid, sulfuric acid, hydrochloric acid and formic acid.

In another embodiment of the present invention, wherein the step (2b) is carried out at temperature between 10° C. to 110° C.

In another embodiment of the present invention, wherein the oxidation in step (3) is carried out by using oxidizing agent selected from the group consisting of iron (III) chloride (FeCl₃) with or without hydrochloric acid (HC₁), hydrogen peroxide (H₂O₂), p-toluenesulfonic acid (p-TSA), acetic acid (AcOH), hydrochloric acid (HCl) and the like; or by using air along with solvent.

In another embodiment of the present invention, wherein the oxidizing agent is used in step (3) is 0.3 to 2 equivalents, preferably 2 equivalents.

In another embodiment of the present invention, wherein the solvent for oxidation in step (3) is comprising of polar protic solvents, aromatic hydrocarbon solvent and aliphatic alcohols.

In another embodiment of the present invention, wherein the volume of the polar protic solvent is used in range 0.3 to 2 volume preferable 1 volume.

In another embodiment of the present invention, wherein the purification of prothioconazole in step (3) is carried out by using solvent selected from aromatic hydrocarbons, alcohols, ethers and the like.

In another embodiment of the present invention, wherein the aromatic hydrocarbon solvents used in step (3) is selected from toluene, n-hexane, n-heptane and the like.

In another embodiment of the present invention, wherein the aliphatic alcohols used in Step (3) is selected from methanol, ethanol, isopropyl alcohol and the like.

In another embodiment of the present invention, wherein the ethers used in step (3) is selected from diisopropyl ether, diethyl ether, tetrahydrofuran,1,4-dioxane and the like.

In another embodiment of the present invention, wherein the oxidation in step (3) is carried out at temperature between 20° C. to 50° C.

The preparation of the starting material used in the present invention are well known in prior art. The invention is further illustrated by the following examples, which should not be construed to limit the scope of the invention in anyway.

Example:

1) Preparation of 2-Chloro-1-(1-Chlorocyclopropyl) Ethenone.

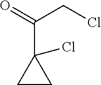

The mixture of 2-acetylbutyrolactone (3.5 Kg, 1.0 eq.) and sulfuryl chloride (3.96 Kg, 1.07 eq.) was stirred below ambient temperature for 1.5 hours (hrs.) under inert atmosphere. After complete conversion, the reaction mixture was further treated with conc. HCl (6.05 Kg, 2.0 eq.) at 70° C. to 100° C. for 3 hrs. The reaction mixture was diluted with water, extracted with dichloromethane (3.5 vol). Organic layer was treated with 48-50% NaOH (2.64 Kg, 1.5 eq.) in presence of tetra n-butyl ammonium bromide (0.035 Kg, 0.005 eq.) below ambient temperature. After completion of reaction, the organic layer was separated by azeotropic distillation. Sulfuryl chloride (3.27 Kg, 1.5 eq.) and methanol (0.3 mole) were added to the organic layer and stirred for 3 hrs. The reaction mixture was quenched with water and layers were separated. Organic layer was washed with saturated sodium bicarbonate (NaHCO₃) solution, brine solution and further distilled to obtain 2-chloro-1-(1-chlorocyclopropyl)ethanone as colorless oil (2.40 kg, 57% yield, GC purity 92.36%); ¹H NMR (CDCl₃, 400 MHz) δ=1.51 (ddd, J=3.6, 4.4, 5.6 Hz, 2H), 1.72 (ddd, J=3.6, 4.4, 5.6 Hz, 2H), 4.91 (s, 3H); ¹³C NMR (DMSO-d6, 400 MHz) δ=21.71, 45.35, 48.14, 197.13; GC-MS: C₅H₆Cl₂O^{+: (M-}1)^{30 :}152.0.

2) Preparation of 2-[2-(1-Chloro-Cyclopropyl)-3-(2-Chloro-Phenyl)-2-Hydroxy-Propyl]-1,2,4-Triazolidine-3-Thione.

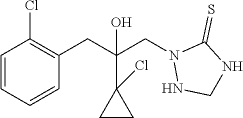 [SMILES: OC(Cc1ccccc1Cl)(CN1NCNC1=S)C1(Cl)CC1]

In reaction flask, magnesium metal (0.68 Kg, 1.7 eq.), iodine (I₂) (0.0025 Kg, 0.0006 eq.) in THF (0.5 L, 0.2 V) and toluene (2.0 L, 0.8 V) mixture (1:4 ratio) were charged. To the reaction mixture, 2-chlorobenzyl chloride (3.42 Kg, 1.3 eq.) in THF (2.5 L, 1.0 V) and toluene (7.5 L, 3.0 V) (1:3) was added and maintained the resulting mixture at 30° C. to 80° C. for 1 hr. 2-Chloro-1-(1-chlorocyclopropyl) ethanone (2.5 Kg, 1 eq.) was added to the Grignard reagent mixture at −5° C. to 30° C. and the reaction mixture was maintained at 40° C. to 55° C. for 3 hrs. 7% aq. HCl (8.25 L, 3.3 vol.) was added to the reaction mixture and layers were separated. The organic layer was washed with water and distilled partially to obtain mixture of 1-chloro-2-(1-chloro-cyclopropyl)-3-(2-chloro-phenyl)-propan-2-ol) and (2-(2-chloro-benzyl)-2-(1-chloro-cyclopropyl)-oxirane (4.6 kg). Further 1.63 Kg (1.0 eq.) of obtained mixture and 76.05% aqueous hydrazine hydrate (1.41 Kg, 3.2 eq.) in n-butanol (n-BuOH) (4.89 L, 3.0 vol) were heated at 60° C. to 100° C. for 1 hr. The reaction mixture was cooled to room temperature and washed with brine solution. The organic layer was treated with 36.06% aq. formaldehyde (HCHO) solution (0.7 Kg, 1.2 eq.), NaSCN (0.65 Kg, 1.2 eq.) and aq. solution of sodium hydrogen sulfate (NaHSO₄) (1.39 Kg, 1.5 eq.) in water (1.95 L, 1.2 V) at 15° C. to 35° C. for 1 hr. The reaction mixture was diluted with water, heptane and further cooled to 5° C. to 10° C. The reaction mixture was filtered and the obtained solid cake was washed with water and dried to obtain 2-[2-(1-chloro-cyclopropyl)-3-(2-chloro-phenyl)-2-hydroxy-propyl]-1,2,4-triazolidine-3-thione as pale yellow to off-white solid (1.58 Kg; 48% yield); ¹H NMR (DMSO-d6, 400 MHz) δ=0.65-0.81 (m, 2H), 0.82-0.94 (m, 1H), 1.08-1.22 (m, 1H), 3.08 (d, J=14 Hz, 1H), 3.40 (d, J=14 Hz, 1H), 3.96 (s, 2H), 4.34 (d, J=9.6 Hz, 2H), 5.08 (s, 1H), 6.20 (t, J=11.2, 1H), 7.18-7.32 (m, 2H), 7.37-7.42 (m, 1H), 7.46-7.59 (m, 1H), 8.84 (m, 1H); ¹³C NMR (DMSO-d6, 400 MHz) δ=10.62, 10.79, 37.23, 46.45, 52.57, 60.34, 75.69, 126.23, 128.07, 128.98, 133.40, 134.51, 181.00; LC-MS: (C₁₄H₁₇N₃Cl₂OS)^{+: (M-}1)⁺346.0 and 348.0.

3) Preparation of Prothioconazole

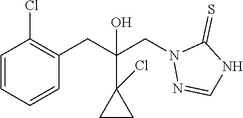 [SMILES: OC(Cc1ccccc1Cl)(Cn1nc[nH]c1=S)C1(Cl)CC1]

To the mixture of 2-[2-(1-chloro-cyclopropyl)-3-(2-chloro-phenyl)-2-hydroxy-propyl]-1, 2,4-triazolidine-3-thione (1.15 Kg, 1.0 eq.) in toluene (9.2 L, 8.0 vol) and isopropyl alcohol (2.3 L, 2.0 vol), the conc. HCl (0.08 Kg, 0.2 eq.) and a solution of FeCl₃.6H₂O (1.83 Kg, 2.08 eq.) in water (5.75 Kg, 5.0 vol.) were added at 20° C. to 30° C. The reaction mixture was stirred for 3 hrs. The reaction mixture was filtered and separated the layers. The organic layer was washed with water and brine solution. The organic layer was partially distilled and heptane (1.15 L, 1.0 vol) was added to the residual mixture at 60° C. to 70 C and stirred at for 1 h. The resulting mixture was cooled to 5° C. to 10° C. and maintained for 1 h. The precipitated solid was filtered, washed with toluene: heptane mixture (0.6 Kg, 0.5 vol), dried to obtain pure Prothioconazole as a off-white to white solid (0.897 Kg, 88.82% yield, HPLC purity>99%; ¹H NMR (DMSO-d6, 400 MHz) δ=0.67-0.74 (m, 2H), 0.77-0.91 (m, 2H), 3.16-3.20 (1H, m), 3.18 (d, J=14 Hz, 1H), 3.34 (d, J =14 Hz, 1H), 4.48 (s, 2H), 5.08 (s, 1H), 7.19-7.33 (m, 2H), 7.36-7.46 (m, 1H), 7.51-7.62 (m, 1H), 8.47 (s, 1H), 13.73 (s, 1H); ¹³C NMR (DMSO, TMS, 400 MHz) δ=10.74, 10.88, 37.65, 46.15, 53.18, 76.38, 126.30, 128.20, 129.07, 133.29, 134.08, 134.55, 139.29, 165.82; LC-MS: (C₁₄H₁₅N₃Cl₂OS)^{+:} 344.0.

1. A process for preparation of Prothioconazole of formula (I) comprising the steps of: 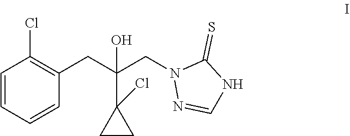 [SMILES: OC(Cc1ccccc1Cl)(Cn1nc[nH]c1=S)C1(Cl)CC1]
1) obtaining compound of formula (III) from a compound of formula (II) by sequential reaction transformations; 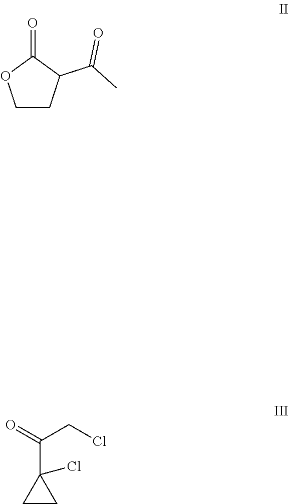
2) obtaining compound of formula (VI) by 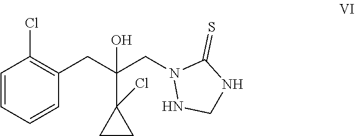 [SMILES: OC(Cc1ccccc1Cl)(CN1NCNC1=S)C1(Cl)CC1]
a) reacting compound of formula (III) with Grignard reagent of formula (IVa), where X is Cl, Br, or I, in solvent to obtain a mixture of oxirane formula (Va) and alcohol formula (Vb) at suitable temperature; 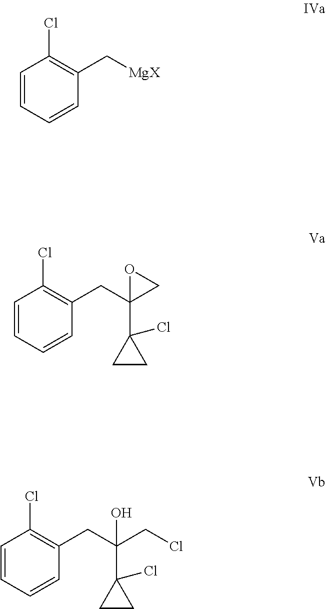
b) reacting mixture of formula (Va) and formula (Vb) with hydrazine hydrate in solvent; followed by reacting with formaldehyde, thiocyanate of formula YSCN, where Y is sodium, potassium or ammonium in presence of acid and water; and
3) obtaining compound of formula (I) by oxidation of compound of formula (VI) in a solvent; optionally the compound is purified.

2. The process as claimed in claim 1, wherein the said sequential reaction transformations of step (1) includes chlorination, hydrolysis, cyclization, and chlorination.

3. The process as claimed in claim 2, wherein the chlorination reaction is carried out using chlorinating agent, with or without catalytic amount of alcohol and in presence or absence of solvent.

4. The process as claimed in claim 3, wherein said chlorinating agent is selected from a group consisting of sulfuryl chloride (SO₂Cl₂), chlorine gas (Cl₂ gas), chlorine gas in presence of sodium acetate (AcONa) in acetic acid (AcOH), thionyl chloride (SOCl₂), N-chloro succinimide (NCS), cyanuric chloride [(NCCl)₃, 1,3-dichloro 5,5-dimethylhydantoin; oxidative chlorinating agents such as oxalyl chloride [(COCl)₂], phosphoryl chloride (POCl₃), phosphorus pentachloride (PCl₅) and phosphorus trichloride (PCl₃).

5. The process as claimed in claim 3, wherein solvent used for chlorination is selected from dichloromethane (DCM), ethylene dichloride (EDC), chloroform (CHCl₃), carbon tetrachloride (CCl₄), toluene, cyclohexane; monohalobenzenes such as monochlorobenzene, dihalobenzenes such as dichlorobenzene, dialkyl (C₁-C₁₂) ethers and water.

6. The process as claimed in claim 3, wherein the alcohol used in chlorination reaction is C₁-C₄ alcohols selected from methanol, ethanol, isopropanol and n-butanol in 0.1 to 3.0 equivalents.

7. The process as claimed in claim 2, wherein hydrolysis is carried out in presence of one or more acids, which is selected from hydrochloric acid (HCl), hydrobromic acid (HBr), sulfuric acid, trifluoroacetic acid (TFA), formic acid (HCOOH) and acetic acid (AcOH).

8. The process as claimed in claim 2, wherein cyclization is carried out using base, which is selected from the group consisting of sodium hydroxide (NaOH), potassium hydroxide (KOH), potassium carbonate (K₂CO₃), sodium carbonate (Na₂CO₃), ammonia (NH₃), ammonium hydroxide (NH₄OH), magnesium tertiary butoxide [(t-BuO) ₂Mg], potassium tertiary butoxide (t-BuOK), and sodium tertiary butoxide (t-BuONa).

9. The process as claimed in claim 2, wherein cyclization is carried out in presence or absence of a phase transfer catalyst, where the phase transfer catalyst is selected from the group consisting of tetra alkyl ammonium halide such as tetrabutylammonium bromide (TBAB), tetrabutylammonium iodide (TBAI), tetrabutylammonium chloride (TBACl ), tetrabutylammonium fluoride (TBAF), benzyltriethyl ammonium chloride; methyl tri alkyl ammonium halides such as methyltricapryl ammonium chloride, methyl tributyl ammonium chloride, methyl trioctyl ammonium chloride ; Aliquat 336 and potassium iodide (KI).

10. The process as claimed in claim 1, wherein the solvent used in step 2 (a) is selected from group consisting of mixture of tetrahydrofuran (THF) and toluene; inert organic solvents such as aliphatic, alicyclic and aromatic hydrocarbons solvents selected from cyclohexane, methylcyclohexane, xylene, benzene, 2-methyltetrahydrofuran, methyl tert-butyl ether, isopropyl ether, dimethoxyethane, dimethoxymethane, 1,3-dioxane, 1,4-dioxane, ethylene glycol diethyl ether, diethylene glycol dimethyl ether, diethylene glycol diethyl ether, diethylene glycol dibutyl ether, polyethylene glycol dimethyl ether, cyclic and acyclic ethers.

11. The process as claimed in claim 1, wherein in step 2(a) is carried out in presence of initiator, which is selected from Iodine, methyl iodide and 1,2-dibromoethane; and said suitable temperature is −5° C. to 80° C.

12. The process as claimed in claim 1, wherein the solvent used in step 2(b) is selected from C₁-C₄ alcohols selected from methanol, ethanol, isopropanol and n-butanol; cyclohexanol, toluene, acetonitrile (ACN), N, N-dimethyl formamide (DMF) and sulfolane.

13. The process as claimed in claim 1, wherein the acid used in step 2(b) is selected from sodium hydrogen sulfate (NaHSO₄), p-toluene sulfonic acid (p-TSA), acetic acid, sulfuric acid, hydrochloric acid and formic acid.

14. The process as claimed in claim 1, wherein the oxidation in step 3 is carried out by using oxidizing agent which is selected from the group consisting of iron (III) chloride (FeCl₃) with or without hydrochloric acid (HCl), hydrogen peroxide (H₂O₂), p-toluene sulfonic acid (p-TSA), acetic acid (AcOH), hydrochloric acid (HCl) and air.

15. The process as claimed in claim 1, wherein the solvent used for oxidation and purification in step 3 is selected from group of aromatic hydrocarbon selected from toluene, n-hexane, n-heptane, aliphatic alcohols selected from methanol, ethanol, isopropyl alcohol; ethers selected from diisopropyl ether, diethyl ether, tetrahydrofuran and 1,4-dioxane.
